(19) United States
(12) Patent Application Publication
(10) Pub. No.: US 2008/0097190 A1
(43) Pub. Date: Apr. 24, 2008

(54) Slitted marker

(76) Inventors: Dirk Hornscheidt, Berlin (DE); Frank Kniep, Grossbeeren (DE); Mario Kilka, Goyatz (DE); Chris Neumann, Berlin (DE)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 11/584,434

(22) Filed: Oct. 20, 2006

(51) Int. Cl. A61B 5/05

(52) U.S. Cl. 600/421

(57) ABSTRACT

A surgical instrument, in particular a marking instrument for marking body tissue, in particular tumor tissue, comprising a hollow needle for insertion into body tissue which encloses a lumen which is radially delimited by an inside wall of the hollow needle and which has an open mouth at the distal needle end, and at least one longitudinally displaceable element guided in the lumen, with distal end portions which have a pre-bend such that the distal end portions are elastically prestressed in the straightened condition and curve in the relaxed condition, wherein the distal end portions of the longitudinally displaceable element are to be pushed starting from a position of being retracted into the lumen, in which the distal end portions are straightened, out of the open distal mouth of the lumen and in so doing curve, wherein the longitudinally displaceable element is formed by a wire having a longitudinal slit, wherein the longitudinal slit extends from a base point at its proximal slit end to an open distal slit end and separates the distal end portions of the longitudinally displaceable element from each other.

### CROSS REFERENCE TO RELATED APPLICATION

Reference is made to and priority claimed from German application Ser. No. 10 2005 053 464.3 filed 07 Nov. 2005.

### BACKGROUND OF THE INVENTION

1. Technical Field

The invention concerns a surgical instrument, in particular a marker instrument for marking body tissue portions. The instrument is intended to be suitable in particular for marking tumor tissue prior to surgical removal of that tissue.

2. Discussion of Related Art

Marking instruments of that kind are basically known. Thus for example both such an instrument and also the use thereof are described in DE 44 24 394 A1.

A component part of the instrument is a hollow needle for insertion into body tissue. The hollow needle encloses a lumen which is open at least at its distal end and generally also at its proximal end. In the radial direction the lumen is defined by an inside wall of the hollow needle.

A further component part of the surgical instrument is an element which is guided in the lumen and which is longitudinally displaceable with respect to the hollow needle. That longitudinally displaceable element has distal end portions which have a pre-bend such that they are elastically prestressed in the straightened condition and curve in the relaxed condition. In that respect the arrangement of the longitudinally displaceable element is such that its pre-bent distal end portions are to be pushed selectively out of the open distal mouth opening of the lumen and in that situation adopt a curved configuration or are to be retracted into the lumen and in that situation are straightened.

As mentioned an instrument of that kind serves for marking body tissue. For that purpose firstly the hollow needle with the wire retracted into the lumen is inserted into the tissue to be marked. The wire is then advanced in the hollow needle so that the prestressed distal end portions which are straightened in the lumen issue distally from the hollow needle, from the open mouth of the lumen. The pre-bend of the distal end portions provides that, when they issue from the open mouth of the lumen, they curve in the direction which is determined by the pre-bend and in that way are anchored in the tissue to be marked. If the surgical instrument is to be re-positioned or removed the longitudinally displaceable element can also be retracted into the hollow needle. In that way the pre-bent distal end portions are straightened again in the lumen. The diameter of the lumen of the hollow needle is not substantially larger than the outside diameter of the elongated element in the straightened condition thereof.

The demands which are made on a marking instrument of that kind include simple and reliable handling as well as the wish to be able to definedly mark a volume of tissue. The marking instrument must be exactly positionable for the last-mentioned purpose. In addition the marking element together with the longitudinally displaceable element should be re-positionable as far as possible. At the same time the longitudinally displaceable element should provide a retaining force which is as great as possible in the tissue, by means of its pre-bent distal end portions. The longitudinally displaceable element should be as easy as possible to advance in the hollow needle. The outside diameter of the needle should be as far as possible <1 mm. And finally, the instrument must also be inexpensive to manufacture as a disposable instrument. The aim of developing a corresponding marking element is therefore to embody the various and in part contradictory properties as well as possible and bring them into harmony with each other.

Known marking elements like that disclosed in DE 44 24 394 leave something to be desired in regard to those demands. Thus it has been found that manufacture is not a simple matter and handling is often not entirely satisfactory.

Therefore the object of the invention is to provide a surgical instrument and in particular a marker instrument which affords a better compromise in regard to the above-mentioned properties.

### DISCLOSURE OF INVENTION

In accordance with the invention the, above-mentioned object is attained by a surgical instrument of the above-described kind, in which the longitudinally displaceable element is formed by a wire with a longitudinal slit. The longitudinal slit extends from a base point at a proximal slit end to an open distal slit end and in that way separates the distal end portions of the longitudinally displaceable element from each other. Unlike the situation with known marking elements in which the longitudinally displaceable element is formed by individual wires which are twisted together and which respectively form a pre-bent distal end portion, the longitudinally displaceable element in the marking instrument according to the invention is formed by a single wire which in a distal longitudinal portion is slitted in the longitudinal direction so that the longitudinally displaceable element is in one piece at least in its distal region.

The invention is based on the realization that the handling properties of known marking elements depend very greatly on manufacturing tolerances. Experiments with a large number of different solutions have finally surprisingly shown that the marking instrument according to the invention is not only less critical in relation to production tolerances but in addition also has very good properties.

It has proven to be advantageous if the pre-bend of the distal end portions only begins at a spacing from the base point of the longitudinal slit so that the distal end portions are unbent in the region of their origin beside the proximal end of the slit. It is possible in that way to effectively prevent fatigue phenomena in respect of the longitudinally displaceable element in the region of the base point of the longitudinal slit so that the reliability of the surgical instrument is increased. In that respect the pre-bend is of such a nature that, on issuing from the open mouth of the lumen, the distal end portions bend away from the longitudinal slit and thus away from each other.

The longitudinal slit preferably extends at least over the greatest part of its length along the longitudinal axis of the longitudinally displaceable element or parallel to that axis. That arrangement provides distal end portions which are of a uniform cross-section over the greatest part of their length. If the longitudinal slit extends along the longitudinal axis of the longitudinally displaceable element and thus divides the longitudinally displaceable element centrally at least over the greatest part of its length, the distal end portions are of an identical cross-sectional shape over the greatest part of their length.

If the pre-bend of those distal end portions differs from each other, that affords the advantage that it makes it possible to distinguish various, differently oriented, longitudinally displaceable elements.

In a preferred variant of the surgical instrument the distal end portions are of differing lengths. That is preferably implemented by the longitudinal slit extending inclinedly with respect to the longitudinal axis of the longitudinally displaceable element in the region of the distal end of the longitudinal slit so that it terminates in an open slit end in the periphery of the longitudinally displaceable element and the longer one of the distal end portions of the longitudinally displaceable element continues distally of the open end of the slit. In the longitudinal portion, disposed distally of the open end of the slit, of the longer one in the distal end, the longer distal end is preferably of a cross-section which corresponds to the cross-section of the longitudinally displaceable element in a longitudinal portion proximally of the base point of the longitudinal slit. A longitudinally displaceable element of that kind can not only be produced in an advantageous manner by a wire of uniform diameter being provided with a suitable slit. In addition a surgical instrument with such a longitudinally displaceable element also has advantageous properties in terms of its handling and in particular the last, unslitted distal end portion of the longer distal end can be pointed in such a way that, together with the pointed end of the hollow needle, it form a closed tip geometry in which the longitudinally displaceable element, in its position of being retracted into the hollow needle, projects with its unslitted tip slightly from the distal end of the hollow needle. In that way the tip of the hollow needle is supported by the tip of the longitudinally displaceable element and accordingly can afford a hollow needle of smaller wall thickness and thus smaller outside diameter. In that case the hollow needle can even be a blunt hollow needle as the tip of the longitudinally displaceable element ultimately forms the tip of the surgical instrument when it is inserted into body tissue. The hollow needle can also comprise plastic material and can thus be for example magnetic resonance-compatible.

In all variants the slit is preferably produced by laser cutting or wire erosion. Wire erosion is a spark erosion process with a wire as a counterpart pole to the longitudinally displaceable element which is to be slitted. The slit width is preferably between 0.03 and 0.1 mm. The outside diameter of the longitudinally displaceable element is preferably between 0.4 mm and 0.6 mm and crucially influences the desired rigidity, which is felt by the physician, when advancing the longitudinally displaceable element. The hollow needle is preferably of an outside diameter of less than 1.2 mm or better 1 mm.

The distal end portions of the longitudinally displaceable element are preferably pointed in order to permit easy penetration of the distal end portions into the body tissue when the longitudinally displaceable element is pushed distally out of the open mouth of the lumen. In that case it has proven to be advantageous if the distal end portions have an inclined chamfer or grind. The inclined ground or chamfer surface produced in that way preferably extends at an acute angle relative to the longitudinal axis of the respective distal end portion and is preferably so arranged that it faces inwardly respectively when the distal end portions are straight.

In a preferred variant the longitudinally displaceable element has two longitudinal slits which extend in different longitudinal planes and which provide that the longitudinally displaceable element has more than two distal end portions, in a preferred variant for example four distal end portions. For that purpose the longitudinal slits preferably extend in mutually perpendicular planes which preferably intersect on the longitudinal axis of the longitudinally displaceable element.

The longitudinally displaceable element is preferably a wire of a superelastic material, in particular a superelastic metal alloy like Nitinol. Nitinol is a known nickel-titanium alloy which has superelastic properties.

In preferred variants the hollow needle can comprise titanium, a titanium alloy or a biocompatible steel alloy. The latter provides that the entire surgical instrument is suitable for use in conjunction with magnetic resonance tomographs (MR-compatible). The use of titanium as the material for the hollow needle is usually problematical because of the roughness of titanium as the roughness excessively impedes pushing the longitudinally displaceable element out. In the case of longitudinally displaceable elements of the claimed kind however the roughness of titanium is innocuous.

Alternative advantageous materials for the hollow needle are MR-compatible plastic materials, in particular polyetheretherketone (PEEK) or carbon fiber-reinforced plastic materials.

The surface of the longitudinally displaceable element is preferably roughened up for example by etching and in that way is better visible in terms of ultrasound observation.

The pre-bend of the distal end portions is preferably such that, in the relaxed condition, the distal end portions assume a curvature of at least 180°. That provides for secure anchoring of the surgical instrument in the body tissue to be marked. As already mentioned hereinbefore the pre-bend of the various distal end portions of a longitudinally displaceable element can be different, whereby in surgical use a distinction in respect of various longitudinally displaceable elements of basically identical geometry becomes possible by virtue of differing orientation of their distal end portions in the tissue.

It has been found that such a pre-bend is retained even after the longitudinally displaceable element has been extended and retracted again a number of times, if the distal end portions are pre-bent firstly with a tighter radius and through a larger angle than corresponds to the desired permanent final geometry and the distal end portions are then straightened once again. After that straightening operation the free ends assume a geometry which they then retain over a large number of load cycles in the form of being pushed out and retracted again.

Preferably the longitudinally displaceable element is formed by a wire, the overall length of which in the straightened condition of the pre-bent distal end portions is such that a proximal end of the wire still projects from a proximal end of the hollow needle even when the pre-bent distal end portions of the wire have been pushed out of the distal mouth opening of the lumen of the hollow needle. Provided at the proximal end of the wire there are preferably two longitudinal markings which are at a longitudinal spacing from each other which corresponds to the dimension by which the longitudinally displaceable element is advanced from its position of being completely retracted into the lumen to a longitudinal position in which the pre-bent distal end portions are pushed out of the open distal mouth of the lumen of the hollow needle.

In a preferred variant, provided at the proximal end of the surgical element is a handle which is preferably releasably connected to the proximal end of the hollow needle and which includes a slider which is releasably connected to the proximal end of the longitudinally displaceable element. The slider is on the one hand reciprocatingly movable between two longitudinal positions and is so connected to the longitudinally displaceable element that, in an end position of the slider, the longitudinally displaceable element is completely retracted into the lumen of the hollow needle while the second end position of the slider corresponds to that longitudinal position of the longitudinally displaceable element in which the pre-bent distal end portions of the longitudinally displaceable element are pushed completely out of the open distal mouth of the lumen of the hollow needle. Such a handle with slider simplifies handling of the surgical instrument because a user no longer has to pay heed to longitudinal markings on the longitudinally displaceable element in order to set the respective correct longitudinal position of the longitudinally displaceable element with respect to the hollow needle. Markings on the longitudinally displaceable element are thus superfluous. In addition the handle makes it easier to advance the wire.

Instead of a slider it is also possible to provide another sliding element which for example is to be reciprocated between longitudinal positions by a push mechanism in the manner of a ballpoint pen.

In a preferred variant the slider can be moved into a third position in which the slider is separated from the longitudinally displaceable element so that the hollow needle is to be moved by means of the handle independently of the longitudinally displaceable element. The latter is required for example if the longitudinally displaceable element is positioned at a desired location in the body tissue of a patient and is fixed by advancing the pre-bent distal end portions and the hollow needle is to be removed without at the same time also removing the longitudinally displaceable element. Finally the longitudinally displaceable element is to remain as a marking element at the intended position in the body tissue of the patient.

The handle and the slider on the handle are preferably of such a configuration, in regard to the last-mentioned variant, that a user must overcome a perceptible resistance when moving the slider from the first or second longitudinal position into the third position. In that way a user can reciprocatingly move the slider between the first and second longitudinal positions practically without any resistance in order to extend the longitudinally displaceable element from the open distal mouth of the hollow needle or to retract the longitudinally displaceable element completely into the hollow needle. If then finally the longitudinally displaceable element is to be released the user must move the slider beyond a perceptible resistance in order to do that.

In a preferred variant there is provided a latching means which latches the slider in the first end position or the second end position respectively so that the longitudinally displaceable wire retains its respective first or second longitudinal position.

Finally it is advantageous if the slider is to be moved into a fourth position and in that position causes the handle to be separated from the hollow needle.

The handle represents an inventive concept which is also to be embodied independently of the specific configuration of the longitudinally displaceable element. Thus the handle can also be used for example in conjunction with such longitudinally displaceable elements which are formed by two wires which are twisted together.

In order to facilitate positioning and in particular re-positioning of the longitudinally displaceable element in the body tissue of a patient, the longitudinally displaceable element can be provided with a sheath which is to be extended from or retracted into the lumen of the hollow needle, together with the longitudinally displaceable element. The function of the sheath is initially to hold the pre-bent distal end portions of the longitudinally displaceable element together so that they do not assume their pre-curved shape as soon as they are pushed out of the open distal mouth of the lumen. That makes it easier to retract the longitudinally displaceable element together with the sheath from body tissue even without the hollow needle in order to re-position the longitudinally displaceable element. It is only when the longitudinally displaceable element is finally moved to its definitive position that the sheath can also be retracted with respect to the longitudinally displaceable element so that the pre-bent distal end portions of the longitudinally displaceable element curve in the intended manner and are anchored in the body tissue.

In an alternative variant the longitudinally displaceable element is an implant which can be pushed completely out of the lumen. After such an implant has once been pushed out of the lumen, it generally cannot be retracted into the lumen again so that, after placement of the implant, re-positioning of the implant is generally no longer possible. It is also possible to provide a releasable coupling between a respective implant and for example a slider wire as referred hereinafter, which coupling permits retraction and thus re-positioning.

In order to push an implant of the last-mentioned kind out of the lumen of the surgical instrument, there is preferably provided a pusher or slider wire which is also guided longitudinally displaceably in the lumen proximally of the longitudinally displaceable element and which is of such a configuration that the longitudinally displaceable element as an implant is to be pushed by means of the slider wire out of the mouth opening of the lumen at least so far that the implant can dig with its pre-bent distal end portions into the tissue in such a way that the implant remains in the tissue upon retraction of the hollow needle.

Instead of a single implant it is also possible for a plurality of implants to be arranged in succession in a magazine-like fashion in the longitudinal direction of the lumen, which implants can then be pushed individually out of the lumen by means of the slider wire. For that purpose the slider wire can have longitudinal markings which, in the longitudinal direction of the slider wire, are at a spacing from each other which corresponds to the straight length of a respective individual implant in the lumen. That makes it possible for example for a physician to advance the slider wire in each case by precisely the amount that is required to deliver a respective implant.

The inside wall of the hollow needle can be provided at least in portion-wise manner with a slip coating which contains a biocompatible organic lubricant. A particularly suitable lubricant is a long-chain wax. The lubricant preferably has a melting point of over 50° C.

It will be noted that an advantage of the longitudinally displaceable element in its configuration according to the invention is that a slip coating is almost superfluous for the reason that the friction between the longitudinally displaceable element and the hollow needle is slight in any case.

If nonetheless a slip coating is provided, it is advantageous for it to be arranged on the inside wall of the hollow needle and not for example externally on the longitudinally displaceable element, for the reason that the first variant affords two advantages: on the one hand the introduction of lubricant into the body tissue can be minimized, while on the other hand that arrangement ensures that sufficient lubricant is available over the entire displacement length of the longitudinally displaceable element. More specifically, if the lubricant were arranged on the outside of the longitudinally displaceable element, then, in the forward movement of the longitudinally displaceable element, the lubricant could be successively removed at the parts with which the longitudinally displaceable element touches the inside wall of the hollow needle.

### BRIEF DESCRIPTION OF THE DRAWINGS

Further advantageous configurations will be apparent from the appendant claims, which are not the subject of comment here, and from the description hereinafter.

The invention will now be described in greater detail by means of an embodiment with reference to the Figures in which:

FIG. 1 shows the essential component parts of a simple embodiment of the surgical instrument according to the invention,

FIGS. 2a through g show the distal end of the surgical element of FIG. 1 in various operating conditions,

FIG. 3 shows a view and a detail view of a first variant of a longitudinally displaceable element of the surgical instrument of FIGS. 1 and 2 with straightened distal end portions,

FIGS. 4a and b show the longitudinally displaceable element of FIG. 3 with relaxed distal end portions in their curved final geometry,

FIGS. 5a through d show the longitudinally displaceable element of FIG. 4 in two production steps,

FIGS. 6a and b show an alternative longitudinally displaceable element for a surgical instrument similar to FIG. 1, wherein the distal end portions of the longitudinally displaceable element are shown in the straightened condition,

FIG. 7 shows a surgical instrument similar to FIG. 1 with a handle at the proximal end with a slider integrated therein for moving the longitudinally displaceable element,

FIGS. 8a and b show a surgical instrument in which the longitudinally displaceable element has a sleeve which is to be removed as desired, and

FIG. 9 shows a surgical instrument in which the longitudinally displaceable element is in the form of an implant which is to be pushed completely out of the lumen.

### DETAILED DESCRIPTION

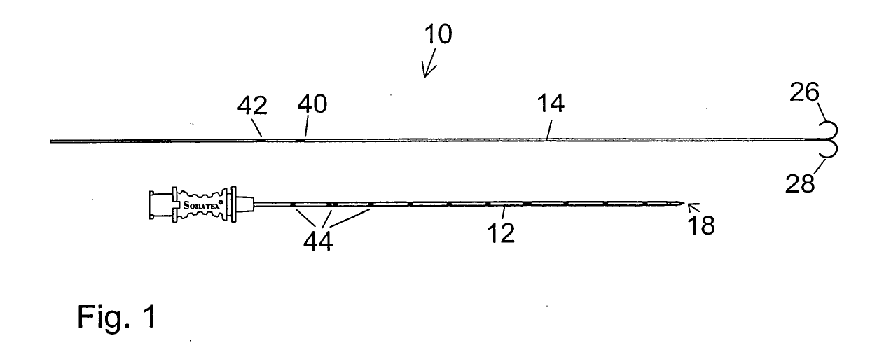

Essential component parts of the surgical instrument 10 in FIG. 1 are a hollow needle 12 and a longitudinally displaceable wire 14 as the longitudinally displaceable element. The hollow needle 12 has a lumen 16 which is open at both longitudinally ends and which is of an inside diameter which is slightly larger than an outside diameter of the longitudinally displaceable wire 14. A distal end 18 of the hollow needle 12 is pointed for insertion into body tissue. There, the lumen 16 has an open distal mouth.

The longitudinally displaceable wire 14 is slitted lengthwise in the region of its distal end 20 by a slit 22, thereby affording two free distal end portions 26 and 28 in the illustrated embodiment.

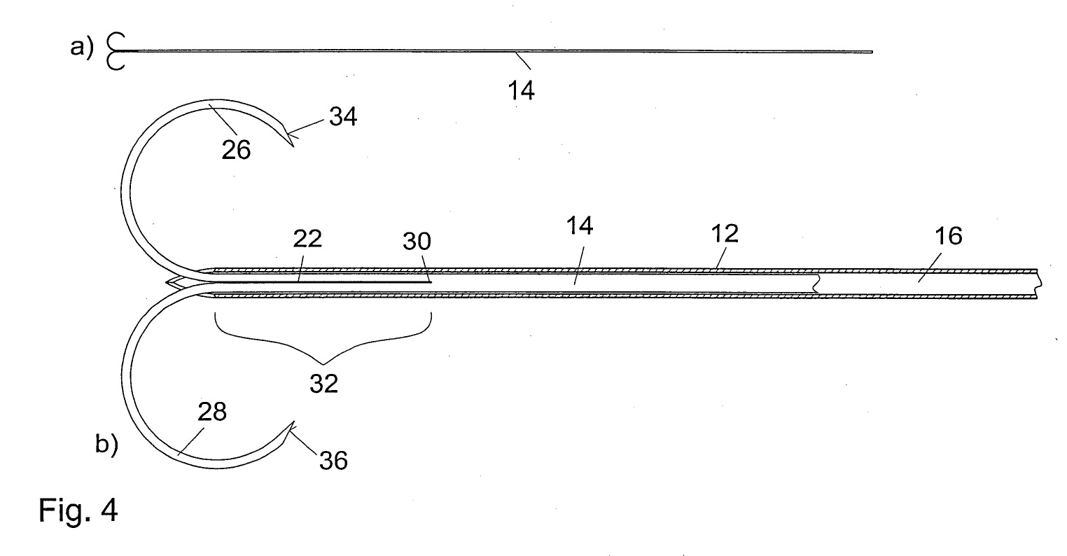

As can be seen from FIGS. 4a and b, in particular FIG. 4b, the distal end portions 26 and 28 are bent through a circular arc segment of about 210°. In that respect, the bend of the distal end portions 26 and 28 does not already start at the base point 30 of the slit 22. Rather, a proximal longitudinal portion 32 of the free distal end portions 26 and 28 is unbent.

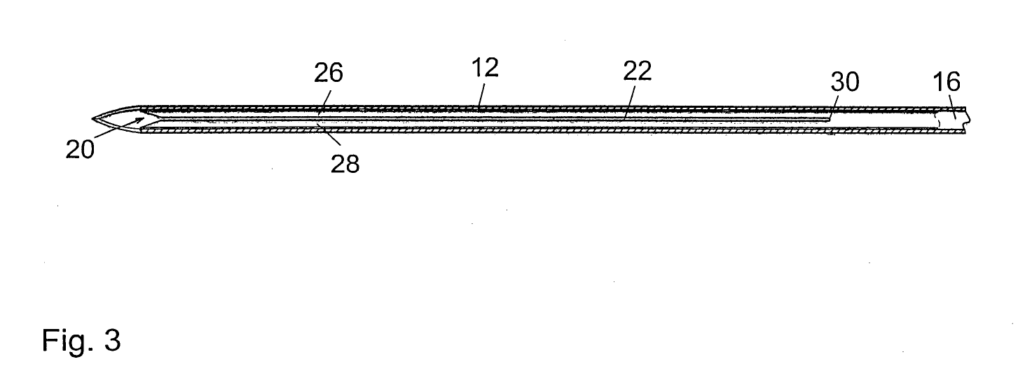

The distal end portions 26 and 28 are pointed, more specifically by a grind which extends at an acute angle of 20° with respect to the longitudinal axis of the distal end portions 26 and 28. That provides ground surfaces 34 and 36 which in the straight condition of the distal end portions 26 and 28 (see FIG. 3) face towards each other.

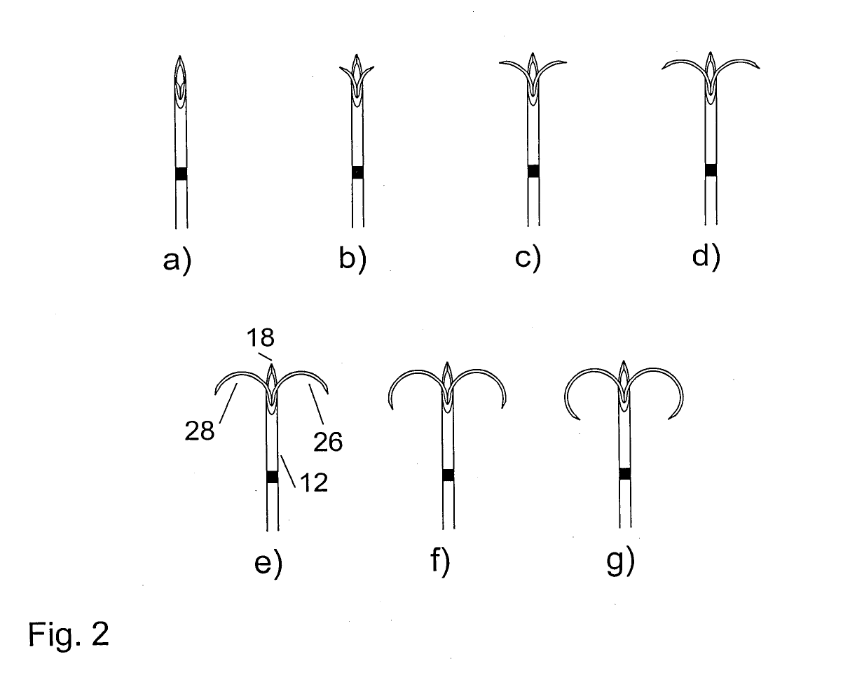

As can be seen from FIGS. 2a through g, when the hollow needle 12 is inserted into body tissue, the longitudinally displaceable wire 14 is initially positioned in such a way that its distal end portions 26 and 28 are retracted entirely into the lumen 16 of the hollow needle (FIG. 2a). When then the longitudinally displaceable wire 14 is advanced in the distal direction of the hollow needle 12 and thus the distal end portions 26 and 28 are pushed out of the open distal mouth of the lumen 16, the distal end portions 26 and 28 curve, as is shown in FIGS. 2b through 2g. Finally, FIG. 2g shows the condition in which the pre-curved portions of the distal end portions 26 and 28 have been pushed completely out of the lumen 16 and have assumed their final geometry in the relaxed condition. Between the condition shown in FIG. 2a and the condition shown in FIG. 2g the longitudinally displaceable wire 14 has thus been advanced with respect to the hollow needle 12 by a length which corresponds to the straightened length of the bent portions of the distal end portions 26 and 28.

The longitudinally displaceable wire 14 bears two markings 40 and 42 which are at a longitudinal spacing from each other, which precisely corresponds to the length by which the longitudinally displaceable wire 14 is advanced between the condition shown in FIG. 2a and that shown in FIG. 2g. The markings 40 and 42 thus enable the physician to establish that the longitudinally displaceable wire 14 has been pushed out of the distal open mouth of the lumen 16 of the hollow needle 12 to such an extent that the pre-bent portions of the distal end portions 26 and 28 are disposed entirely outside the lumen 16.

Markings 44 are also disposed on the outside of the hollow needle 12, which enable the physician to detect the depth of penetration of the hollow needle 12 into body tissue.

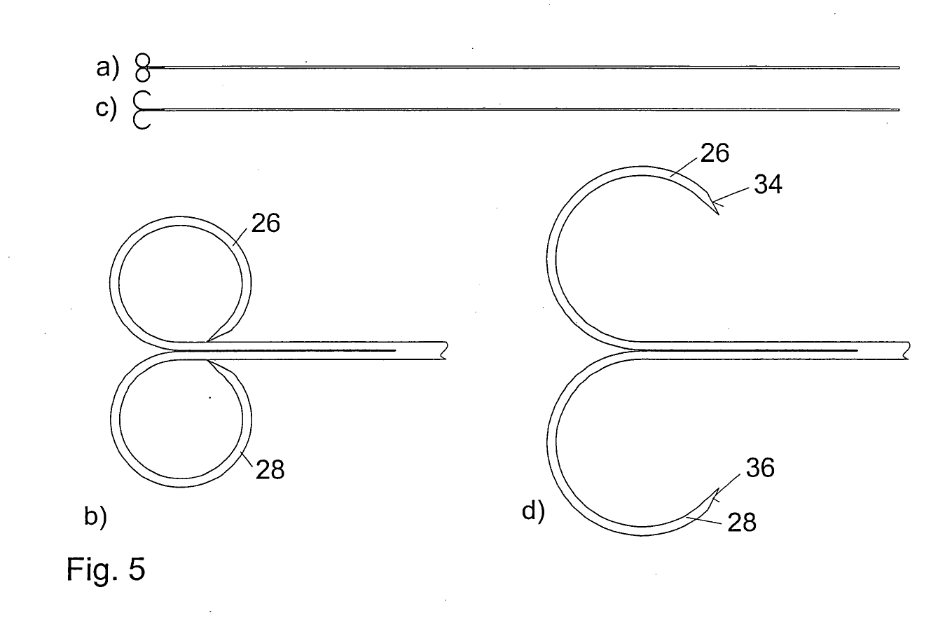

In order to produce the final geometry as shown in FIG. 4 and also FIG. 5d for the pre-bent portions of the distal end portions 26 and 28, the portions which are separated from each other by the slit 22 are firstly curved through a tighter radius than corresponds to the final geometry. In that respect the portions, which are tightly pre-curved in that way, of the distal end portions 26 and 28 extend through a circular angle segment of between about 330° and 340°; see FIG. 5b. Then the portions, which are pre-bent in that way, of the distal end portions 26 and 28 are straightened and then assume the final geometry shown in FIGS. 4b and 5d which the distal end portions retain even after multiple straightening and then relaxation (curving).

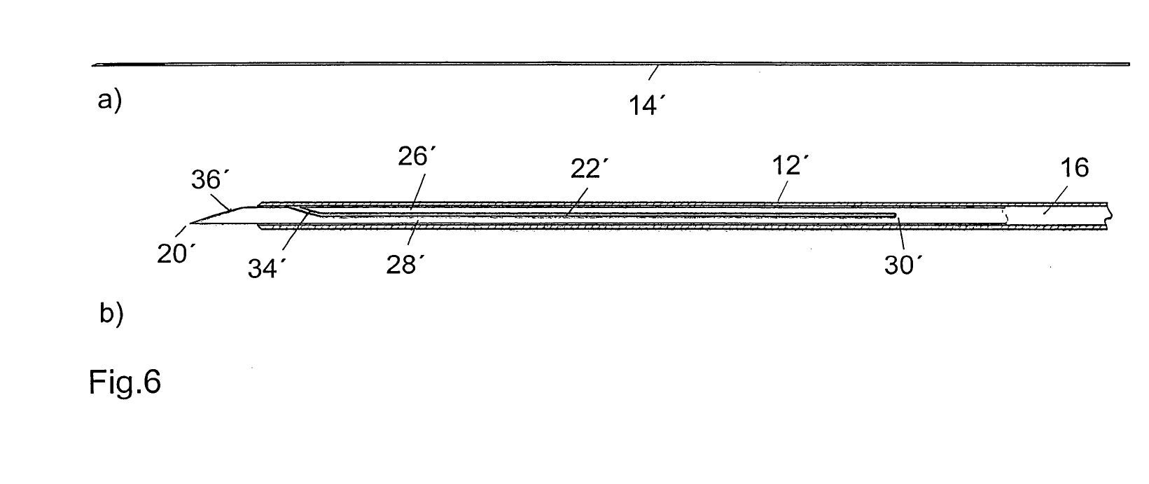

FIG. 6 shows an alternative longitudinally slitted wire 14′ in the straightened condition. As can be seen from FIG. 6 the slit 22′ does not open entirely at the distal tip of the longitudinally displaceable wire 14′ but opens proximally of the distal end 20 laterally in the circumference of the longitudinally displaceable wire 14′. That affords two distal end portions 26′ and 28′, which are of differing lengths, of the longitudinally displaceable wire 14′.

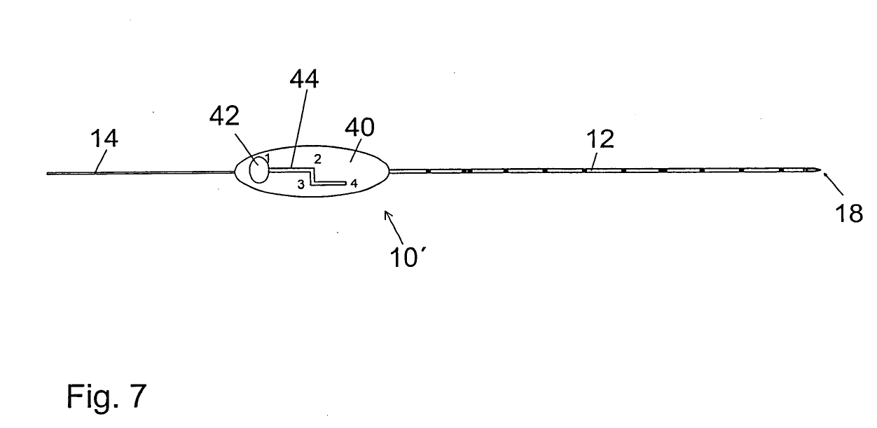

FIG. 7 shows an alternative surgical instrument 10′ in which a handle 40 is fixed to the proximal end of the hollow needle. The handle 40 has a slider 42 which is connected as a sliding element to the longitudinally displaceable wire 14. The longitudinally displaceable wire 14 can be displaced between two end positions in the longitudinal direction of the hollow needle by means of the slider 42 serving as a sliding element. In the first end position which is identified on the handle 40 by “1”, the distal end of the longitudinally displaceable wire 14 is retracted completely into the hollow needle 12. In that position, the hollow needle can be inserted into body tissue. When the distal end 18 of the hollow needle 12 is at the desired location in the body tissue, the slider 42 can be moved into the second position which is identified on the handle 40 by “2”. In that second end position, the curved end portions of the longitudinally displaceable wire 14 have been pushed completely out of the mouth opening at the distal end 18 of the hollow needle 12.

In order to avoid inadvertent actuation of the slider 42 or to prevent the longitudinally displaceable wire 14 being displaced of its own accord, the slider 42 latches in engagement in the two end positions “1” and “2”.

When finally the curved distal end portions of the longitudinally displaceable wire 14 have been pushed out of the hollow needle 12 at the distal end 18 at the desired location in the body tissue and the longitudinally displaceable wire 14 is to remain at the desired location, the longitudinally displaceable wire 14 can be separated from the sliding element and thus from the slider 42 by the slider 42 being moved into the position identified by “3” on the handle 40. In position “3” of the slider 42 the handle 40, the slider 42 and also the hollow needle 12 are separated from the longitudinally displaceable wire 14 and can be retracted from the body tissue while the longitudinally displaceable wire 14 remains in the body tissue.

Finally, in the illustrated preferred embodiment of the surgical instrument 10′, there is also a fourth position for the slider 42 which is identified on the handle 40 by “4”, in which the handle 40 is to be separated from the hollow needle 12. In that way, when the longitudinally displaceable wire 14 has been moved into position, it is possible for the handle 40 to be separated from the longitudinally displaceable wire 14 and the hollow needle 12 so that if necessary the hollow needle 12 together with the longitudinally displaceable wire 14 can remain inserted into body tissue while the handle 40 is removed.

As can be seen from FIG. 7, provided for the slider 42 is a sliding guide 44 which delimits the longitudinal movement of the slider 42 between the end positions “1” and “2” of the longitudinally displaceable wire 14 and requires a sideways movement of the slider 42 in order to release the sliding element connected to the slider 42 from the longitudinally displaceable wire 14. The sideways movement of the slider between the positions “2” and “3” which is required to release the longitudinally displaceable wire requires a resistance to be overcome, so that the slider 42 cannot be inadvertently moved into the position “3”. That prevents inadvertent release of the longitudinally displaceable wire 14 from the slider 42.

In an alternative variant (not shown) the slider is only to be reciprocatingly moved between the positions “1” and “2”. One or two separate additional actuating elements can be provided for releasing the handle from the longitudinally displaceable wire or from the hollow needle.

It has already been noted at another point herein that a handle 40 is not limited to use with a longitudinally displaceable element 14 or 14′ of the kind claimed herein, but is also to be used with other longitudinally displaceable elements, for example in the form of two or more wires which are twisted together. The internal mechanical structure of the handle 40 is to be embodied by a person skilled in the art in various ways which are known to him, in such a way as to afford the above-described functionality. In accordance with an alternative configuration of the handle (not shown here), it has a push element in the manner of a ballpoint pen, with which a sliding element is to be reciprocatingly moved by a push movement between the two end positions for the longitudinally displaceable wire.

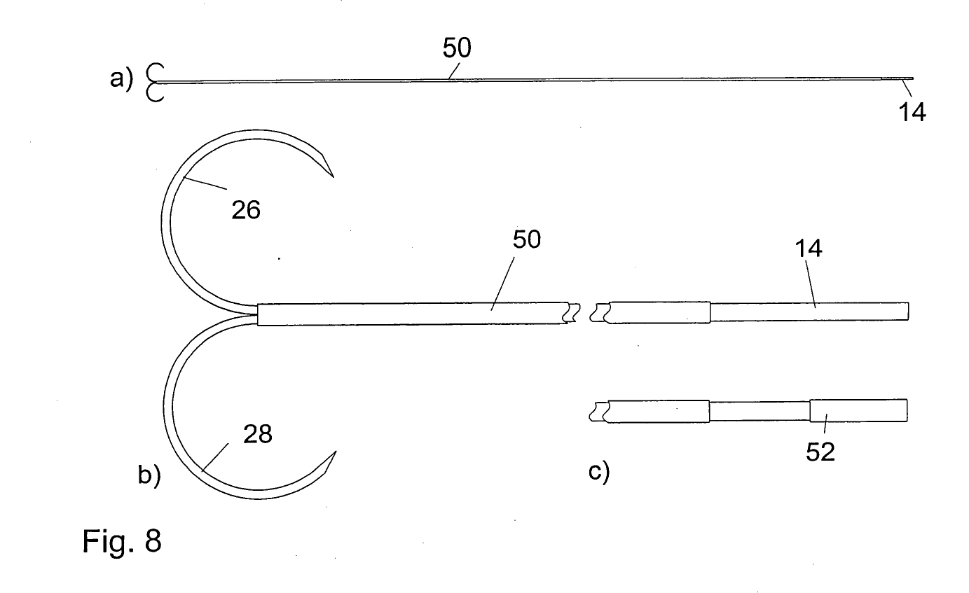

FIG. 8 shows a longitudinally displaceable wire 14 of the kind shown in FIGS. 1 through 5, which is enclosed by a sheath 50. The sheath 50 is longitudinally displaceable with respect to the longitudinally displaceable wire 14. The purpose of the sheath 50, in a position which is not shown in FIG. 8, is to hold the two pre-bent distal end portions 26 and 28 in the straightened condition by the sheath enclosing the distal end portions 26 and 28 over the entire length thereof. The longitudinally displaceable wire 14, together with the sheath 50 in a position of having been advanced as far as the distal end 20 of the longitudinally displaceable wire 14, can be inserted into the lumen 16 of the hollow needle 12 and, together with the sheath 50, pushed distally out of the mouth opening at the distal end 18 of the hollow needle 12. In that situation the sheath 50 initially prevents the pre-bent distal end portions 26 and 28 from spreading and assuming a curved configuration. It is only when the sheath 50 is retracted in the direction of the proximal end of the longitudinally displaceable wire 14 that the distal end portions 26 and 28 are released and can assume the curved configuration.

The sheath only needs to be retracted to the extent shown in FIGS. 8a and b. Therefore, after spreading of the distal end portions 26 and 28, the sheath 50 together with the longitudinally displaceable wire 14 can remain in the body tissue of the patient and allows the longitudinally displaceable wire to be removed from the body tissue again, even without the assistance for example of a hollow needle, by the sheath 50 being held fast and by the longitudinally displaceable wire 14 being retracted with respect to the sheath. The fact that in that case the distal end portions 26 and 28 are retracted into the sheath 50 and as a result are straightened prevents body tissue being unnecessarily damaged upon removal of the longitudinally displaceable wire 14. When the distal end portions 26 and 28 are completely retracted into the sheath 50 and thereby straightened, the sheath 50 together with the longitudinally displaceable wire 14 can be pulled out of the body tissue without further risk of injury.

As shown in FIG. 8c, in the proximal end of the longitudinally displaceable wire 14 an abutment element 52 can be fixedly connected to the longitudinally displaceable wire 14. The abutment element 52 limits the displacement travel of the longitudinally displaceable wire 14 with respect to the sheath 50 in the distal direction so that, admittedly at least the distal end portions 26 and 28 are to be pushed completely out the distal end of the sheath 50, but the sheath 50 is not inadvertently pulled off beyond the proximal end of the longitudinally displaceable wire 14.

It should be pointed out at this juncture that the sheath 50 in combination with a longitudinally displaceable wire 14′ of the kind shown in FIG. 6 can perform the function of the hollow needle 12 and can thus replace same. In that sense a sheath is also a variant of a hollow needle in the sense of the invention. As already explained at another point herein, the tip at the distal end 20′ of the longitudinally displaceable wire 14′ is suitable for permitting the surgical element to be inserted into body tissue without a pointed hollow needle being required for that purpose.

The sheath 50 preferably comprises sufficiently stiff plastic material. It can however also be formed by a thin metal tube.

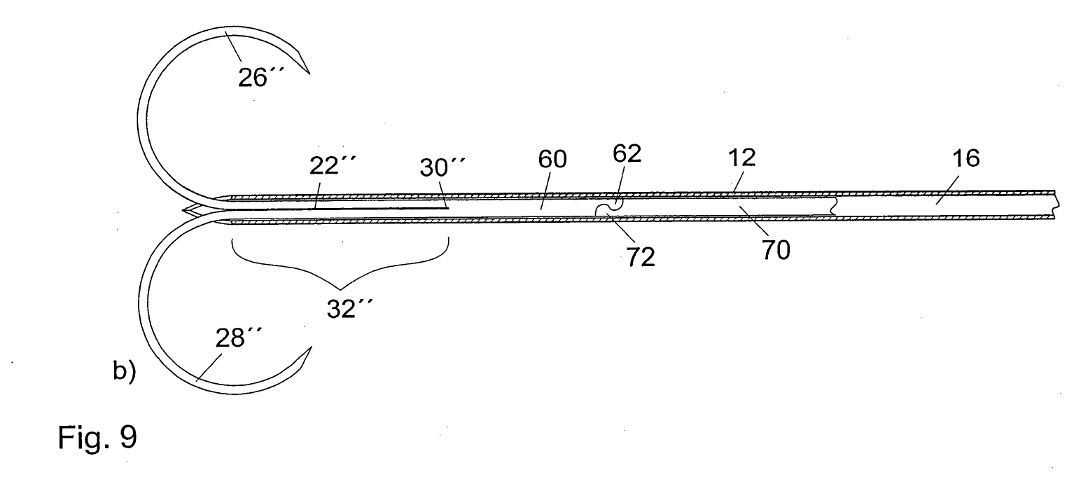

FIG. 9 shows a surgical instrument in which, unlike the situation with the previously illustrated variants, a longitudinally displaceable element 60 is to be pushed completely out of the open mouth at the distal end of the lumen 16 of a hollow needle. A slider wire 70 proximally adjoining the longitudinally displaceable element 60 serves for that purpose. A distal end of the slider wire 70 and the proximal end of the longitudinally displaceable element 60 are in contact with each other in the region of a releasable coupling 62, 72 formed by a projecting nose 62 at the proximal end of the longitudinally displaceable element 60, which nose 62 engages behind a corresponding nose 72 at the distal end of the slider wire 70, in relation to the longitudinal direction of the longitudinally displaceable element 60. As long as the coupling formed by the two noses 62 and 72 between the longitudinally displaceable element 60 and the slider wire 70 is disposed in the lumen 16, the longitudinally displaceable element 60 can still be retracted into the lumen 16 by means of the slider wire 70 even if the pre-bent distal end portions 26″ and 28″ of the longitudinally displaceable element 60 are already pushed out of the open mouth at the distal end of the lumen 16, as in the condition shown in FIG. 9.

The longitudinally displaceable element 60 can be completely implanted by being pushed completely, including its nose 62 at the proximal end, out of the lumen 16. It will be appreciated that the coupling formed by the two noses 62 and 72 is then no longer in the engaged condition and it is then no longer possible for the longitudinally displaceable element 60 to be pulled back.

In regard to the other features the longitudinally displaceable element 60 corresponds to the longitudinally displaceable element 14 shown in FIGS. 1 through 5.

The slider wire 70, like the longitudinally displaceable element 60, can comprise titanium or a titanium alloy such as Nitinol. Alternatively a biocompatible steel alloy is also a suitable material for the slider wire 70.

1. A surgical instrument, in particular a marking instrument for marking body tissue, in particular tumor tissue, comprising
a hollow needle (12) for insertion into body tissue which encloses a lumen (16) which is radially delimited by an inside wall of the hollow needle and which has an open mouth at the distal needle end (18), and
at least one longitudinally displaceable element (14; 14′; 60) guided in the lumen,
with distal end portions (26; 28; 26′, 28′, 26″, 28″) which have a pre-bend such that the distal end portions are elastically prestressed in the straightened condition and curve in the relaxed condition,
wherein the distal end portions (26; 28; 26′, 28′, 26″, 28″) of the longitudinally displaceable element (14; 14′; 60) are to be pushed starting from a position of being retracted into the lumen, in which the distal end portions are straightened, out of the open distal mouth of the lumen (16) and in so doing curve,
characterized in that the longitudinally displaceable element is formed by a wire having a longitudinal slit (22; 22′; 22″) wherein the longitudinal slit (22; 22′; 22″) extends from a base point (30; 30′; 30″) at its proximal slit end to an open distal slit end and separates the distal end portions (26, 28; 26′, 28′; 26″, 28″) of the longitudinally displaceable element (14; 14′; 60) from each other.

2. A surgical instrument as set forth in claim 1 characterized in that the pre-bend of the distal end portions (26, 28; 26′, 28′; 26″; 28″) begins only at a spacing (32) in the distal direction from the base point (30; 30′; 30″) and faces away from the longitudinal slit (22; 22′; 22″).

3. A surgical instrument as set forth in claim 1 characterized in that the longitudinal slit (22; 22′; 22″) extends at least over the greatest part of its length along or parallel to the longitudinal axis of the longitudinally displaceable element (14; 14′; 60).

4. A surgical instrument as set forth in one of claim 1 characterized in that the distal end portions (26′, 28′) of the longitudinally displaceable element (14′) are of a different length.

5. A surgical instrument as set forth in claim 4 characterized in that a distal slit portion of the longitudinal slit (22′) extends inclinedly relative to the longitudinal axis of the longitudinally displaceable element (14′) so that the distal slit end is disposed laterally in the circumference of the straightened longitudinally displaceable element (14′) and in that way the longitudinal slit separates a shorter distal end portion (26′) of the longitudinally displaceable element and a longer distal end portion (28′) of the longitudinally displaceable element (14′) from each other.

6. A surgical instrument as set forth in claim 5 characterized in that the cross-sectional area of the longer distal end portion (28′), in portions laterally adjoining the longitudinal slit is smaller than in portions disposed immediately distally of the distal slit end.

7. A surgical instrument as set forth in claim 6 characterized in that, in its unslitted portion disposed immediately distally of the distal slit end, the longer distal end portion (28′) is of a cross-sectional area which corresponds to the cross-sectional area of the longitudinally displaceable element (14′) in its unslitted portion immediately proximally of the base point (30′) of the longitudinal slit (22′).

8. A surgical instrument as set forth in claim 4 characterized by a hollow needle (12) of plastic material.

9. A surgical instrument as set forth in claim 1 characterized by a hollow needle (12) of titanium or a titanium-bearing material.

10. A surgical instrument as set forth in claim characterized in that the distal end portions (26, 28; 26′, 28′; 26″, 28″) of the longitudinally displaceable element (14) are pointed.

11. A surgical instrument as set forth in claim 10 characterized in that the distal end portions (26, 28; 26′, 28′; 26″, 28″) have an inclined grind or chamfer.

12. A surgical instrument as set forth in claim 11 characterized in that a respective distal end portion (26, 28; 26′, 28′; 26″, 28″) has an inclined ground or chamfer surface (34, 36; 34′, 36′; 34″, 36″) which extends at an acute angle relative to a center line of a respective distal end portion (26, 28; 26′, 28′; 26″, 28″).

13. A surgical instrument as set forth in claim 11 characterized in that a respective ground surface (34, 36; 34′, 36′; 34″, 36″) or chamfer surface is arranged facing inwardly when the longitudinally displaceable wire (14; 14′; 60) is retracted into the lumen (16).

14. A surgical instrument as set forth in claim 1 characterized in that the longitudinally displaceable element has two longitudinal slits which extend in different longitudinal planes, thereby providing four distal end portions.

15. A surgical instrument as set forth in claim 1 characterized in that the longitudinally displaceable element (14; 14′; 60) is formed by a wire of a superelastic material, in particular a superelastic metal alloy such as Nitinol.

16. A surgical instrument as set forth in claim 1 characterized by a pre-bend of the distal end portions (26; 28; 26′, 28′; 26″, 28″) in such a way that the distal end portions in the relaxed condition assume a curvature of at least 180°.

17. A surgical instrument as set forth in claim 1 characterized in that the longitudinally displaceable element is in the form of an implant (60) which is to be pushed completely out of the lumen.

18. A surgical instrument as set forth in claim 17 characterized in that the surgical instrument has a slider wire (70) which proximally of the longitudinally displaceable element (60) is longitudinally displaceably guided in the lumen and is so adapted that the longitudinally displaceable element (60) as an implant is to be pushed out of the distal mouth of the lumen by means of the slider wire (70) sufficiently far to cause the implant to be able to dig sufficiently in body tissue to be marked so that the implant remains in the body tissue when the hollow needle together with the slider wire is withdrawn.

19. A surgical instrument as set forth in claim 1 characterized in that fixed to a proximal end of the hollow needle (12) is a handle having a sliding element which is releasable connected to the longitudinally displaceable element and which is reciprocatingly movable between two longitudinal positions of which a first longitudinal position corresponds to a condition of the surgical element in which the distal end portions (26, 28; 26′, 28′) or at least a shorter one (26′) of the distal end portions are or is completely retracted into the lumen (16) of the hollow needle (12) while a second longitudinal position corresponds to a condition of the surgical element in which at least the curved longitudinal portions of the distal end portions (26, 28; 26′, 28′) project out of the open mouth at the distal end of the lumen.

20. A surgical instrument as set forth in claim 19 characterized in that the sliding element is movable into a third position in which the sliding element and therewith the handle are released from the longitudinally displaceable element (14; 14′).

21. A surgical instrument as set forth in claim 1 characterized in that the longitudinally displaceable element is surrounded by a sheath which is longitudinally displaceable with respect to the longitudinally displaceable element and which is to be pushed out the lumen (16) of the hollow needle (12) together with the longitudinally displaceable element.

22. A surgical instrument as set forth in claim 21 characterized in that the sheath comprises plastic material.
